(19) United States
(12) Patent Application Publication
(10) Pub. No.: US 2005/0065166 A1
(43) Pub. Date: Mar. 24, 2005

(54) Substituted pyrimidines

(76) Inventors: Ernst Rudolf F. Gesing, Erkrath (DE); Mark Wilhelm Drewes, Langenfeld (DE); Peter Dahmen, Neuss (DE); Dieter Feucht, Kelkheim (DE); Rolf Pontzen, Leichlingen (DE)

Correspondence Address: BAYER CROPSCIENCE LP;Patent Department, 100 BAYER ROAD, PITTSBURGH, PA, 15205-9741, US

(21) Appl. No.: 10/493,781

(22) PCT Filed: Oct. 21, 2002
(86) PCT No.: PCT/EP02/11744

(30) Foreign Application Priority Data
Nov. 2, 2001 (DE) 101 54 075.2

(51) Int. Cl. A61K 31/517; A61K 31/519; A61K 31/513

(52) U.S. Cl. 514/258.1; 514/269; 544/315; 544/253; 514/266.3

(57) ABSTRACT

The invention relates to substituted pyrimidines of the general formula (I) 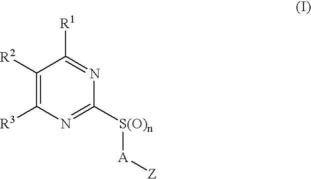
in which n, R¹, R², R³ and Z are as defined in the disclosure, to their use as crop treatment agents, in particular as herbicides, and to processes for their preparation.

The invention relates to novel substituted pyrimidines, to a process for their preparation and to their use as crop treatment agents, in particular as herbicides.

It is already known that certain substituted pyrimidines, such as, for example, the compound 5-(3,5-bistrifluoromethylphenyl)-3-[1-(4,5,6-trimethylpyrimid-2-ylthio)-ethyl]-1,2,4-oxadiazole, have herbicidal properties (cf. GB-A-2205101). However, the action of these compounds is not entirely satisfactory.

Further substituted pyrimidines, such as, for example, the compounds 2-benzylthio-4-chloro-5-methylpyrimidine, 4-chloro-2-(2,4-dichlorobenzylthio)-5-methylpyrimidine, 4-chloro-2-(2-chlorobenzylthio)-5-methylpyrimidine and 4-chloro-2-(4-chlorobenzylthio)-5-methylpyrimidine (cf. J. Org. Chem. 27 (1962), 181-185), the compound 4,6-dichloro-5-methyl-2-(naphthalen-2-ylmethylthio)pyrimidine (cf. WO-A-95/13267), the compound 5-methyl-2-[(1-methyl-5-nitro-1H-imidazol-2-yl)methylthio]pyrimidine (cf. U.S. Pat. No. 3,991,191), and the compounds 2-(thien-2-ylmethylsulphonyl)-5-trifluoromethylpyrimidine, 2-phenylmethylthio-5-trifluoromethylpyrimidine, 2-(thien-2-ylmethylthio)-5-trifluoromethylpyrimidine, 2-phenylmethylsulphonyl-5-trifluoromethylpyrimidine and 2-(thien-2-ylmethylsulphinyl)-5-trifluoromethylpyrimidine (cf. GB-A-2135992) have been disclosed as potential pharmaceutically active compounds. However, hitherto nothing has been disclosed relating to possible applications of these compounds in crop protection.

This invention now provides the substituted pyrimidines of the general formula (I) 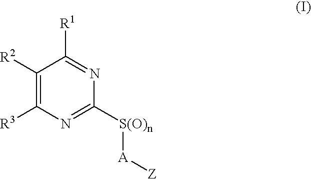
in which
n represents the number 0, 1 or 2,
A represents straight-chain or branched alkanediyl having 1 to 6 carbon atoms,
R¹ represents hydrogen, halogen, optionally halogen- or C₁-C₄-alkoxy-substituted alkyl having 1 to 6 carbon atoms, or optionally halogen- or C₁-C₄-alkyl-substituted phenyl,
R² represents optionally halogen- or C₁-C₄-alkoxy-substituted alkyl having 1 to 6 carbon atoms, represents optionally halogen- or C₁-C₄-alkyl-substituted cycloalkyl having 3 to 8 carbon atoms, or represents optionally halogen- or C₁-C₄-alkyl-substituted phenyl-C₁-C₄-alkyl, or together with R¹ or together with R³ represents optionally C₁-C₄-alkyl-substituted alkanediyl having 3 to 5 carbon atoms or an optionally halogen- or C₁-C₄-alkyl-substituted benzo grouping,
R³ represents hydrogen, halogen, optionally halogen- or C₁-C₄-alkoxy-substituted alkyl having 1 to 6 carbon atoms, or optionally halogen- or C₁-C₄-alkyl-substituted phenyl, and
Z represents in each case optionally substituted phenyl, naphthyl, pyridinyl, pyrimidinyl, furyl, thienyl, oxazolyl or thiazolyl, where the substituents possible in each case are selected from the list below:
   nitro, cyano, carboxyl, carbamoyl, thiocarbamoyl, halogen, in each case optionally cyano-, carboxyl-, carbamoyl-, thiocarbamoyl-, halogen-, C₁-C₄-alkoxy-, C₁-C₄-alkylthio-, C₁-C₄-alkylsulphinyl- or C₁-C₄-alkylsulphonyl-substituted alkyl, alkoxy, alkoxycarbonyl, alkylthio, alkylsulphinyl, alkylsulphonyl, alkylamino, alkylaminocarbonyl or alkylcarbonylamino having in each case 1 to 6 carbon atoms in the alkyl groups, in each case optionally halogen-substituted alkylenedioxy having 1 or 2 carbon atoms, dialkylamino, dialkylaminocarbonyl or dialkylaminosulphonyl having in each case 1 to 4 carbon atoms in the alkyl groups, in each case optionally nitro-, cyano-, carboxyl-, carbamoyl-, thiocarbamoyl-, halogen-, C₁-C₄-alkyl-, C₁-C₄-haloalkyl-, C₁-C₄-alkoxy-, C₁-C₄-haloalkoxy-, C₁-C₄-alkylthio-, C₁-C₄-haloalkylthio-, C₁-C₄-alkylsulphinyl-, C₁-C₄-haloalkylsulphinyl-, C₁-C₄-alkylsulphonyl- or C₁-C₄-haloalkylsulphonyl-substituted phenyl, phenoxy, phenylthio, phenylsulphinyl, phenylsulphonyl, phenylamino, phenylcarbonylamino, phenyl-C₁-C₄-alkyl, phenyl-C₁-C₄-alkoxy, phenoxy-C₁-C₄-alkyl, phenyl-C₁-C₄-alkylthio, phenyl-C₁-C₄-alkylsulphinyl, phenyl-C₁-C₄-alkylsulphonyl, phenyl-C₁-C₄-alkylamino.

Saturated or unsaturated hydrocarbon groupings, such as alkyl, alkanediyl, alkenyl or alkynyl are in each case straight-chain or branched as far as this is possible—including in combination with heteroatoms, such as in alkoxy.

Optionally substituted radicals can be mono- or polysubstituted, and in the case of polysubstitution, the substituents can be identical or different.

The compounds of the general formula (I) according to the invention may contain one or more asymmetrically substituted carbon atoms, in which cases they may be present in different enantiomeric (R- and S-configured) forms or diastereomeric forms. In these cases, the invention relates both to the use of the different possible individual enantiomeric or stereoisomeric forms of the compounds of the general formula (I) and of mixtures of these isomeric compounds.

Preferred substituents or ranges of the radicals present in the formulae given above and below are defined below.

n preferably represents the number 0, 1 or 2.

A preferably represents straight-chain or branched alkanediyl having 1 to 4 carbon atoms.

R¹ preferably represents hydrogen, halogen, optionally halogen- or C₁-C₄-alkoxy-substituted alkyl having 1 to 4 carbon atoms, or optionally halogen- or C₁-C₄-alkyl-substituted phenyl.

R² preferably represents optionally halogen- or C₁-C₄-alkoxy-substituted alkyl having 1 to 5 carbon atoms, represents optionally halogen- or C₁-C₃-alkyl-substituted cycloalkyl having 3 to 7 carbon atoms, or represents optionally halogen- or C₁-C₄-alkyl-substituted phenyl-C₁-C₄-alkyl, or together with R¹ or together with R³ represents optionally C₁-C₃-alkyl-substituted alkanediyl having 3 to 5 carbon atoms or an optionally halogen- or C₁-C₃-alkyl-substituted benzo grouping.

R³ preferably represents hydrogen, halogen, optionally halogen- or C₁-C₄-alkoxy-substituted alkyl having 1 to 4 carbon atoms, or optionally halogen- or C₁-C₄-alkyl-substituted phenyl.

Z preferably represents in each case optionally substituted phenyl, naphthyl, pyridinyl, pyrimidinyl, furyl, thienyl, oxazolyl or thiazolyl, where the substituents possible in each case are preferably selected from the list below:
nitro, cyano, carboxyl, carbamoyl, thiocarbamoyl, halogen, in each case optionally cyano-, carboxyl-, carbamoyl-, thiocarbamoyl-, halogen-, C₁-C₄-alkoxy-, C₁-C₄-alkylthio-, C₁-C₄-alkylsulphinyl- or C₁-C₄-alkylsulphonyl-substituted alkyl, alkoxy, alkoxycarbonyl, alkylthio, alkylsulphinyl, alkylsulphonyl, alkylamino, alkylaminocarbonyl or alkylcarbonylamino having in each case 1 to 5 carbon atoms in the alkyl groups, in each case optionally halogen-substituted alkylenedioxy having 1 or 2 carbon atoms, dialkylamino, dialkylaminocarbonyl or dialkylaminosulphonyl having in each case 1 to 3 carbon atoms in the alkyl groups, in each case optionally nitro-, cyano-, carboxyl-, carbamoyl-, thiocarbamoyl-, halogen-, C₁-C₄-alkyl-, C₁-C₄-haloalkyl-, C₁-C₄-alkoxy-, C₁-C₄-haloalkoxy-, C₁-C₄-alkylthio-, C₁-C₄-haloalkylthio-, C₁-C₄-alkylsulphinyl-, C₁-C₄-haloalkylsulphinyl-, C₁-C₄-alkylsulphonyl- or C₁-C₄-haloalkylsulphonyl-substituted phenyl, phenoxy, phenylthio, phenylsulphinyl, phenylsulphonyl, phenylamino, phenylcarbonylamino, phenyl-C₁-C₄-alkyl, phenyl-C₁-C₄-alkoxy, phenoxy-C₁-C₄-alkyl, phenyl-C₁-C₄-alkylthio, phenyl-C₁-C₄-alkylsulphinyl, phenyl-C₁-C₄-alkylsulphonyl, phenyl-C₁-C₄-alkylamino.

n particularly preferably represents the number 0, 1 or 2.

A particularly preferably represents methylene, ethane-1,1-diyl (ethylidene), ethane-1,2-diyl (dimethylene), propane-1,1-diyl (propylidene), propane-1,2-diyl or propane-1,3-diyl (trimethylene).

R¹ particularly preferably represents hydrogen, fluorine, chlorine, bromine, iodine, represents in each case optionally fluorine-, chlorine-, bromine-, methoxy-, ethoxy-, n- or isopropoxy-substituted methyl, ethyl, n- or isopropyl, n-, iso-, s- or t-butyl or optionally fluorine-, chlorine-, bromine-, methyl-, ethyl-, n- or isopropyl-, n-, iso-, s- or t-butyl-substituted phenyl.

R² particularly preferably represents in each case optionally fluorine-, chlorine-, bromine-, methoxy-, ethoxy-, n- or isopropoxy-substituted methyl, ethyl, n- or isopropyl, n-, iso-, s- or t-butyl, represents in each case optionally fluorine-, chlorine-, methyl- or ethyl-substituted cyclopropyl, cyclobutyl, cyclopentyl or cyclohexyl, or represents in each case optionally fluorine-, chlorine-, bromine-, methyl-, ethyl-, n- or isopropyl, n-, iso-, s- or t-butyl-substituted benzyl or phenylethyl, or together with R¹ or together with R³ represents in each case optionally methyl- and/or ethyl-substituted propane-1,3-diyl (trimethylene) or butane-1,4-diyl (tetramethylene) or an optionally fluorine-, chlorine- or methyl-substituted benzo grouping.

R³ particularly preferably represents hydrogen, fluorine, chlorine, bromine, iodine, in each case optionally fluorine-, chlorine-, bromine-, methoxy-, ethoxy-, n- or isopropoxy-substituted methyl, ethyl, n- or isopropyl, n-, iso-, s- or t-butyl, or optionally fluorine-, chlorine-, bromine-, methyl-, ethyl-, n- or isopropyl-, n-, iso-, s- or t-butyl-substituted phenyl.

Z particularly preferably represents in each case optionally substituted phenyl, naphthyl, pyridinyl, pyrimidinyl, furyl, thienyl, oxazolyl or thiazolyl, where the substituents possible in each case are particularly preferably selected from the following list:
nitro, cyano, carboxyl, carbamoyl, thiocarbamoyl, fluorine, chlorine, bromine, iodine, in each case optionally cyano-, carboxyl-, carbamoyl-, thiocarbamoyl-, fluorine-, chlorine-, methoxy-, ethoxy-, n- or isopropoxy-, methylthio-, ethylthio-, n- or isopropylthio-, methylsulphinyl-, ethylsulphinyl-, methylsulphonyl- or ethylsulphonyl-substituted methyl, ethyl, n- or isopropyl, n-, iso-, s- or t-butyl, methoxy, ethoxy, n- or isopropoxy, n-, iso-, s- or t-butoxy, methoxycarbonyl, ethoxycarbonyl, n- or isopropoxycarbonyl, methylthio, ethylthio, n- or isopropylthio, n-, iso-, s- or t-butylthio, methylsulphinyl, ethylsulphinyl, n- or isopropylsulphonyl, methylsulphonyl, ethylsulphonyl, methylamino, ethylamino, n- or isopropylamino, n-, iso-, s- or t-butylamino, methylaminocarbonyl, ethylaminocarbonyl, n- or isopropylaminocarbonyl, acetylamino, propionylamino, n- or isobutyroylamino, in each case optionally fluorine- and/or chlorine-substituted methylenedioxy or ethylenedioxy, dimethylamino, diethylamino, dimethylaminocarbonyl or dimethylaminosulphonyl, in each case optionally nitro-, cyano-, carboxyl-, carbamoyl-, thiocarbamoyl-, fluorine-, chlorine-, bromine-, methyl-, ethyl-, n- or isopropyl-, n-, iso-, s- or t-butyl-, trifluoromethyl-, methoxy-, ethoxy-, n- or isopropoxy-, difluoromethoxy-, trifluoromethoxy-, methylthio-, ethylthio-, n- or isopropylthio-, difluoromethylthio-, trifluoropmethylthio-, methylsulphinyl-, ethylsulphinyl-, trifluoromethylsulphinyl-, methylsulphonyl-, ethylsulphonyl- or trifluoromethylsulphonyl-substituted phenyl, phenoxy, phenylthio, phenylsulphinyl, phenylsulphonyl, phenylamino, phenylcarbonylamino, benzyl, phenylethyl, phenylmethoxy, phenylethoxy, phenoxymethyl, phenoxyethyl, phenylmethylthio, phenylethylthio, phenylmethylsulphinyl, phenylethylsulphinyl, phenylmethylsulphonyl, phenylethylsulphonyl, phenylmethylamino, phenylethylamino.

n very particularly preferably represents the number 0, 1 or 2.

A very particularly preferably represents methylene, ethane-1,1-diyl (ethylidene) or ethane-1,2-diyl (dimethylene).

R¹ very particularly preferably represents hydrogen, fluorine, chlorine, bromine, in each case optionally fluorine-, chlorine-, bromine-, methoxy- or ethoxy-substituted methyl, ethyl, n- or isopropyl, or optionally fluorine-, chlorine-, bromine- or methyl-substituted phenyl.

R² very particularly preferably represents in each case optionally fluorine-, chlorine-, bromine-, methoxy- or ethoxy-substituted methyl, ethyl, n- or isopropyl, n-, iso- or s-butyl, or represents optionally fluorine-, chlorine-, bromine- or methyl-substituted benzyl, or together with R¹ or together with R³ represents optionally methyl- and/or ethyl-substituted propane-1,3-diyl (trimethylene) or butane-1,4-diyl (tetramethylene) or an optionally fluorine-, chlorine- or methyl-substituted benzo grouping.

R³ very particularly preferably represents hydrogen, fluorine, chlorine, bromine, in each case optionally fluorine-, chlorine-, bromine-, methoxy- or ethoxy-substituted methyl, ethyl, n- or isopropyl, or optionally fluorine-, chlorine-, bromine- or methyl-substituted phenyl.

Z very particularly preferably represents in each case optionally substituted phenyl, pyridinyl, pyrimidinyl, furyl, thienyl, oxazolyl or thiazolyl, where the substituents possible in each case are in particular selected from the following list:
nitro, cyano, carboxyl, carbamoyl, thiocarbamoyl, fluorine, chlorine, bromine, in each case optionally cyano-, carboxyl-, carbamoyl-, thiocarbamoyl-, fluorine-, chlorine-, methoxy-, ethoxy-, n- or isopropoxy-, methylthio-, ethylthio-, n- or isopropylthio-, methylsulphinyl-, ethylsulphinyl-, methylsulphonyl- or ethylsulphonyl-substituted methyl, ethyl, n- or isopropyl, n-, iso-, s- or t-butyl, methoxy, ethoxy, n- or isopropoxy, methoxycarbonyl, ethoxycarbonyl, n- or isopropoxycarbonyl, methylthio, ethylthio, n- or isopropylthio, methylsulphinyl, ethylsulphinyl, methylsulphonyl, ethylsulphonyl, methylamino, ethylamino, n- or isopropylamino, methylaminocarbonyl, ethylaminocarbonyl, n- or isopropylaminocarbonyl, acetylamino, propionylamino, in each case optionally fluorine- and/or chlorine-substituted methylenedioxy or ethylenedioxy, dimethylamino, dimethylaminocarbonyl or dimethylaminosulphonyl, in each case optionally nitro-, cyano-, carboxyl-, carbamoyl-, thiocarbamoyl-, fluorine-, chlorine-, bromine-, methyl-, ethyl-, n- or isopropyl-, n-, iso-, s- or t-butyl-, trifluoromethyl-, methoxy-, ethoxy-, n- or isopropoxy-, difluoromethoxy-, trifluoromethoxy-, methylthio-, ethylthio-, n- or isopropylthio-, difluoromethylthio-, trifluoromethylthio-, methylsulphinyl-, ethylsulphinyl-, trifluoromethylsulphinyl-, methylsulphonyl-, ethylsulphonyl- or trifluoromethylsulphonyl-substituted phenyl, phenoxy, phenylthio, phenylsulphinyl, phenylsulphonyl, phenylamino, phenylcarbonylamino, benzyl, phenylethyl, phenylmethoxy, phenylethoxy, phenoxymethyl, phenoxyethyl, phenylmethylthio, phenylethylthio, phenylmethylsulphinyl, phenylethylsulphinyl, phenylmethylsulphonyl, phenylethylsulphonyl, phenylmethylamino, phenylethylamino.

A further group of compounds according to the invention which may be mentioned are the compounds in which Z represents optionally substituted phenyl, pyridinyl, pyrimidinyl, furyl, oxazolyl or thiazolyl and most preferably optionally substituted phenyl, pyridinyl, pyrimidinyl or thiazolyl, where the substituents are defined as stated in the preferred ranges for Z mentioned above. In this group according to the invention, only the compounds known from J. Org. Chem. 27 (1962), 181-185 are not novel.

The novel substituted pyrimidines of the general formula (I) have strong and selective herbicidal activity.

The novel substituted pyrimidines of the general formula (I) are obtained when,
(a) if in the formula (I) n represents the number 0, mercaptopyrimidines of the general formula (II) 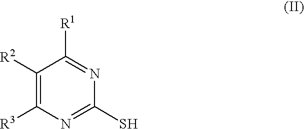
   in which
R¹, R¹ and R³ are as defined above
   are reacted with haloalkyl compounds of the general formula (III) 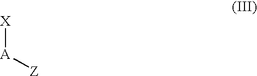
   in which
A and Z are as defined above and
X represents halogen,
   if appropriate in the presence of a reaction auxiliary and if appropriate in the presence of a diluent,
   or when,
(b) if in the formula (I) n represents the number 1 or 2, substituted pyrimidines of the general formula (Ia) 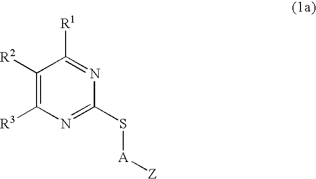
   in which
A, R¹, R²R³ and Z are as defined above
   are reacted with an oxidizing agent, if appropriate in the presence of a catalyst and if appropriate in the presence of a diluent.

Using, for example, 4,5-dimethyl-2-pyrimidinethiol and 4-fluorobenzyl chloride as starting materials, the course of the reaction in the process (a) according to the invention can be illustrated by the following formula scheme:

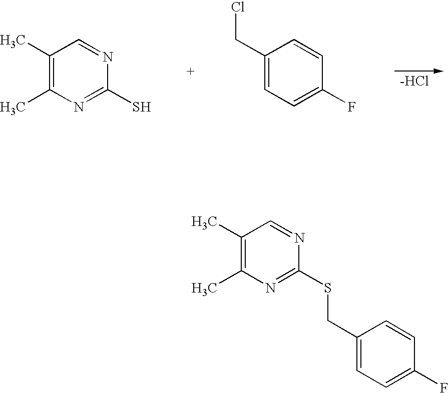

Using, for example, 2-[(4-fluorophenyl)methylthio]-4,5-dimethylpyrimidine and hydrogen peroxide as starting materials, the course of the reaction in the process (b) according to the invention can be illustrated by the following formula scheme:

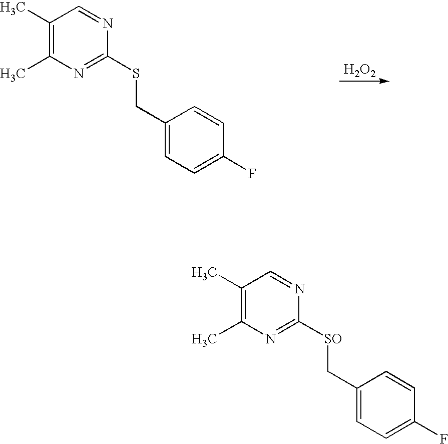

The formula (II) provides a general definition of the mercaptopyrimidines to be used as starting materials in the process (a) according to the invention for preparing compounds of the general formula (I). In the general formula (II), R¹, R² and R³ preferably or in particular have those meanings which have already been mentioned above, in connection with the description of the compounds of the general formula (I) according to the invention, as being preferred or as being particularly preferred for R¹, R² and R³.

The starting materials of the general formula (II) are known and/or can be prepared by processes known per se (cf. Chemiker-Zeitung 101 (1977), 305-307, Chem. Ber. 110 (1977), 2872-2879, J. Chem. Soc., Perkin Trans. 1, 1977, 1688-1692, J. Prakt. Chem. 321 (1979), 619-628, Heterocycles 25 (1987), 393-397, Aust. J. Chem. 45 (1992), 1045-1050, DE-A-2403340, DE-A-2454728, DE-A-2455582, GB-A-2205101).

The formula (III) provides a general definition of the haloalkyl compounds further to be used as starting materials in the process (a) according to the invention for preparing compounds of the general formula (I). In the general formula (III), A and Z preferably or in particular have those meanings which have already been mentioned above, in connection with the description of the compounds of the general formula (I) according to the invention, as being preferred or as being particularly preferred for A and Z; X preferably represents fluorine, chlorine, bromine or iodine, in particular chlorine or bromine.

The starting materials of the general formula (III) are known organic chemicals for synthesis.

The formula (Ia) provides a general definition of the substituted pyrimidines to be used as starting materials in the process (b) according to the invention for preparing compounds of the general formula (I). In the general formula (Ia), A, R¹, R², R³ and Z preferably or in particular have those meanings which have already been mentioned above, in connection with the description of the compounds of the general formula (I) according to the invention, as being preferred or as being particularly preferred for A, R¹, R², R³ and Z.

As novel substances, the starting materials of the general formula (Ia) also form part of the subject-matter of the present application; they can be prepared in accordance with the description of the process (a) according to the invention.

The process (b) according to the invention for preparing the compounds of the formula (I) is carried out using an oxidizing agent. Suitable oxidizing agents are the customary chemicals suitable for oxidizing organic sulphides (thioethers) to the corresponding sulphoxides or sulphones. Examples of suitable oxidizing agents which may be mentioned are: hydrogen peroxide (H₂O₂), performic acid, peracetic acid, perpropionic acid, perbenzoic acid and 3-chloroperbenzoic acid, and also chlorine or hypochlorous acid and its alkali metal or alkaline earth metal salts.

Suitable reaction auxiliaries for the process (a) according to the invention are, in general, the customary inorganic or organic bases or acid acceptors. These preferably include alkali metal or alkaline earth metal acetates, amides, carbonates, bicarbonates, hydrides, hydroxides or alkoxides, such as, for example, sodium acetate, potassium acetate or calcium acetate, lithium amide, sodium amide, potassium amide or calcium amide, sodium carbonate, potassium carbonate or calcium carbonate, sodium bicarbonate, potassium bicarbonate or calcium bicarbonate, lithium hydride, sodium hydride, potassium hydride or calcium hydride, lithium hydroxide, sodium hydroxide, potassium hydroxide or calcium hydroxide, sodium methoxide, ethoxide, n- or isopropoxide, n-, iso-, s- or t-butoxide or potassium methoxide, ethoxide, n- or isopropoxide, n-, iso-, s- or t-butoxide; furthermore also basic organic nitrogen compounds, such as, for example, trimethylamine, triethylamine, tripropylamine, tributylamine, ethyldiisopropylamine, N,N-dimethylcyclohexylamine, dicyclohexylamine, ethyldicyclohexylamine, N,N-dimethylaniline, N,N-dimethylbenzylamine, pyridine, 2-methyl-, 3-methyl-, 4-methyl-, 2,4-dimethyl-, 2,6-dimethyl-, 3,4-dimethyl- and 3,5-dimethylpyridine, 5-ethyl-2-methylpyridine, 4-dimethylaminopyridine, N-methylpiperidine, 1,4-diazabicyclo[2.2.2]octane (DABCO), 1,5-diazabicyclo[4.3.0]non-5-ene (DBN), or 1,8-diazabicyclo[5.4.0]undec-7-ene (DBU).

Further suitable reaction auxiliaries for the processes (a) and (b) according to the invention are also phase-transfer catalysts. Examples of such catalysts which may be mentioned are:

tetrabutylammonium bromide, tetrabutylammonium chloride, tetraoctylammonium chloride, tetrabutylammonium hydrogen sulphate, methyltrioctylammonium chloride, hexadecyltrimethylammonium chloride, hexadecyltrimethylammonium bromide, benzyltrimethylammonium chloride, benzyltriethylammonium chloride, benzyltrimethylammonium hydroxide, benzyltriethylammonium hydroxide, benzyltributylammonium chloride, benzyltributylammonium bromide, tetrabutylphosphonium bromide, tetrabutylphosphonium chloride, tributylhexadecylphosphonium bromide, butyltriphenylphosphonium chloride, ethyltrioctylphosphonium bromide, tetraphenylphosphonium bromide.

The process (a) according to the invention for preparing the compounds of the general formula (I) is preferably carried out using one or more diluents. Suitable diluents for carrying out the process (a) according to the invention are, in addition to water, especially inert organic solvents. These include, in particular, aliphatic, alicyclic or aromatic, optionally halogenated hydrocarbons, such as, for example, benzine, benzene, toluene, xylene, chlorobenzene, dichlorobenzene, petroleum ether, hexane, cyclohexane, dichloromethane, chloroform, carbon tetrachloride; ethers, such as diethyl ether, diisopropyl ether, dioxane, tetrahydrofuran or ethylene glycol dimethyl ether or ethylene glycol diethyl ether; ketones, such as acetone, butanone or methyl isobutyl ketone; nitriles, such as acetonitrile, propionitrile or butyronitrile; amides, such as N,N-dimethylformamide, N,N-dimethylacetamide, N-methylformanilide, N-methylpyrrolidone or hexamethylphosphoric triamide; esters, such as methyl acetate or ethyl acetate; sulphoxides, such as dimethyl sulphoxide; alcohols, such as methanol, ethanol, n- or isopropanol, ethylene glycol monomethyl ether, ethylene glycol monoethyl ether, diethylene glycol monomethyl ether, diethylene glycol monoethyl ether, their mixtures with water or pure water.

The process (b) according to the invention is, if appropriate, carried out in the presence of a catalyst. Suitable catalysts are, preferably, salts of metals of transition group IV., V. and VI. of the Periodic Table of the Elements. Examples which may be mentioned are sodium (meta)vanadate, sodium molybdate and sodium tungstate.

The process (b) according to the invention is preferably carried out using a diluent. Suitable diluents are, in addition to water, organic solvents which are customary for oxidation reactions. These preferably include chlorinated hydrocarbons, such as methylene chloride, ethylene chloride, chloroform, carbon tetrachloride, 1,1,2-trichloroethane, chlorobenzene and o-dichlorobenzene, alcohols, such as methanol, ethanol, propanol, isopropanol, butanol, isobutanol and sec-butanol, carboxylic acids, such as formic acid, acetic acid and propionic acid.

When carrying out the processes (a) and (b) according to the invention, the reaction temperatures can be varied within a relatively wide range. In general, the processes are carried out at temperatures between −20° C. and +150° C., preferably between 0° C. and 100° C.

The processes according to the invention are generally carried out under atmospheric pressure. However, it is also possible to carry out the processes according to the invention under elevated or reduced pressure—in general between 0.1 bar and 10 bar.

For carrying out the processes according to the invention, the starting materials are generally employed in approximately equimolar amounts. However, it is also possible to use a relatively large excess of one of the components. The reaction is generally carried out in a suitable diluent in the presence of a reaction auxiliary or catalyst, and the reaction mixture is generally stirred at the required temperature for a number of hours. Work-up is carried out by customary methods (cf. the Preparation examples).

The active compounds according to the invention can be used as defoliants, desiccants, haulm killers and, especially, as weed killers. Weeds in the broadest sense are understood to mean all plants which grow in locations where they are undesired. Whether the substances according to the invention act as total or selective herbicides depends essentially on the amount used.

The active compounds according to the invention can be used, for example, in connection with the following plants:

Dicotyledonous weeds of the genera: Abutilon, Amaranthus, Ambrosia, Anoda, Anthemis, Aphanes, Atriplex, Bellis, Bidens, Capsella, Carduus, Cassia, Centaurea, Chenopodium, Cirsium, Convolvulus, Datura, Desmodium, Emex, Erysimum, Euphorbia, Galeopsis, Galinsoga, Galium, Hibiscus, Ipomoea, Kochia, Lamium, Lepidium, Lindernia, Matricaria, Mentha, Mercurialis, Mullugo, Myosotis, Papaver, Pharbitis, Plantago, Polygonum, Portulaca, Ranunculus, Raphanus, Rorippa, Rotala, Rumex, Salsola, Senecio, Sesbania, Sida, Sinapis, Solanum, Sonchus, Sphenoclea, Stellaria, Taraxacum, Thlaspi, Trifolium, Urtica, Veronica, Viola, Xanthium.

Dicotyledonous crops of the genera: Arachis, Beta, Brassica, Cucumis, Cucurbita, Helianthus, Daucus, Glycine, Gossypium, Ipomoea, Lactuca, Linum, Lycopersicon, Nicotiana, Phaseolus, Pisum, Solanum, Vicia.

Monocotyledonous weeds of the genera: Aegilops, Agropyron, Agrostis, Alopecurus, Apera, Avena, Brachiaria, Bromus, Cenchrus, Commelina, Cynodon, Cyperus, Dactyloctenium, Digitaria, Echinochloa, Eleocharis, Eleusine, Eragrostis, Eriochloa, Festuca, Fimbristylis, Heteranthera, Imperata, Ischaemum, Leptochloa, Lolium, Monochoria, Panicum, Paspalum, Phalaris, Phleum, Poa, Rottboellia, Sagittaria, Scirpus, Setaria, Sorghum.

Monocotyledonous crops of the genera: Allium, Ananas, Asparagus, Avena, Hordeum, Oryza, Panicum, Saccharum, Secale, Sorghum, Triticale, Triticum, Zea.

However, the use of the active compounds according to the invention is in no way restricted to these genera, but also extends in the same manner to other plants.

The active compounds according to the invention are suitable, depending on the concentration, for the total control of weeds, for example on industrial terrain and rail tracks, and on paths and areas with and without tree plantings. Similarly, the active compounds according to the invention can be employed for controlling weeds in perennial crops, for example forests, decorative tree plantings, orchards, vineyards, citrus groves, nut orchards, banana plantations, coffee plantations, tea plantations, rubber plantations, oil palm plantations, cocoa plantations, soft fruit plantings and hop fields, on lawns, turf and pastureland, and for the selective control of weeds in annual crops.

The compounds of the formula (I) according to the invention have strong herbicidal activity and a broad active spectrum when used on the soil and on above-ground parts of plants. To a certain extent they are also suitable for the selective control of monocotyledonous and dicotyledonous weeds in monocotyledonous and dicotyledonous crops, both by the pre-emergence and by the post-emergence method.

At certain concentrations or application rates, the active compounds according to the invention can also be employed for controlling animal pests and fungal or bacterial plant diseases. If appropriate, they can also be used as intermediates or precursors for the synthesis of other active compounds.

All plants and plant parts can be treated in accordance with the invention. Plants are to be understood as meaning in the present context all plants and plant populations such as desired and undesired wild plants or crop plants (including naturally occurring crop plants). Crop plants can be plants which can be obtained by conventional plant breeding and optimization methods or by biotechnological and recombinant methods or by combinations of these methods, including the transgenic plants and inclusive of the cultivars protectable or not protectable by plant breeders' rights. Plant parts are to be understood as meaning all parts and organs of plants above and below the ground, such as shoot, leaf, flower and root, examples which may be mentioned being leaves, needles, stalks, stems, flowers, fruit bodies, fruits, seeds, roots, tubers and rhizomes. The plant parts also include harvested material, and vegetative and generative propagation material, for example cuttings, tubers, rhizomes, offsets and seeds.

Treatment according to the invention of the plants and plant parts with the active compounds is carried out directly or by allowing the compounds to act on their surroundings, environment or storage space by the customary treatment methods, for example by immersion, spraying, evaporation, fogging, scattering, painting on and, in the case of propagation material, in particular in the case of seeds, also by applying one or more coats.

The active compounds can be converted into the customary formulations such as solutions, emulsions, wettable powders, suspensions, powders, dusts, pastes, soluble powders, granules, suspension-emulsion concentrates, natural and synthetic materials impregnated with active compound, and microencapsulations in polymeric materials.

These formulations are produced in a known manner, for example by mixing the active compounds with extenders, that is, liquid solvents and/or solid carriers, optionally with the use of surfactants, that is, emulsifiers and/or dispersants, and/or foam formers.

If the extender used is water, it is also possible, for example, to use organic solvents as cosolvents. The following are essentially suitable as liquid solvents: aromatics such as xylene, toluene or alkylnaphthalenes, chlorinated aromatics or chlorinated aliphatic hydrocarbons such as chlorobenzenes, chloroethylenes or methylene chloride, aliphatic hydrocarbons such as cyclohexane or paraffins, for example mineral oil fractions, mineral and vegetable oils, alcohols such as butanol or glycol and their ethers and esters, ketones such as acetone, methyl ethyl ketone, methyl isobutyl ketone or cyclohexanone, strongly polar solvents such as dimethylformamide and dimethyl sulphoxide, or else water.

Suitable solid carriers are: for example ammonium salts and ground natural minerals such as kaolins, clays, talc, chalk, quartz, attapulgite, montmorillonite or diatomaceous earth, and ground synthetic materials such as highly-disperse silica, alumina and silicates; suitable solid carriers for granules are: for example crushed and fractionated natural rocks such as calcite, marble, pumice, sepiolite and dolomite, or else synthetic granules of inorganic and organic meals, and granules of organic material such as sawdust, coconut shells, maize cobs and tobacco stalks; suitable emulsifiers and/or foam formers are: for example nonionic and anionic emulsifiers such as polyoxyethylene fatty acid esters, polyoxyethylene fatty alcohol ethers, for example alkylaryl polyglycol ethers, alkylsulphonates, alkyl sulphates, arylsulphonates, or else protein hydrolysates; suitable dispersants are: for example lignosulphite waste liquors and methylcellulose.

Tackifiers such as carboxymethylcellulose and natural and synthetic polymers in the form of powders, granules or latices, such as gum arabic, polyvinyl alcohol and polyvinyl acetate, or else natural phospholipids such as cephalins and lecithins and synthetic phospholipids can be used in the formulations. Other possible additives are mineral and vegetable oils.

It is possible to use colorants such as inorganic pigments, for example iron oxide, titanium oxide and Prussian Blue, and organic colorants such alizarin colorants, azo colorants and metal phthalocyanine colorants, and trace nutrients such as salts of iron, manganese, boron, copper, cobalt, molybdenum and zinc.

The formulations generally comprise between 0.1 and 95% by weight of active compound, preferably between 0.5 and 90%.

For controlling weeds, the active compounds according to the invention, as such or in their formulations, can also be used as mixtures with known herbicides and/or substances which improve the compatibility with crop plants (“safeners”), finished formulations or tank mixes being possible. Also possible are mixtures with weed-killers comprising one or more known herbicides and a safener.

Possible components for the mixtures are known herbicides, for example

acetochlor, acifluorfen (-sodium), aclonifen, alachlor, alloxydim (-sodium), ametryne, amicarbazone, amidochlor, amidosulfuron, anilofos, asulam, atrazine, azafenidin, azimsulfuron, beflubutamid, benazolin (-ethyl), benfuresate, bensulfuron (-methyl), bentazone, benzfendizone, benzobicyclon, benzofenap, benzoylprop (-ethyl), bialaphos, bifenox, bispyribac (-sodium), bromobutide, bromofenoxim, bromoxynil, butachlor, butafenacil (-allyl), butroxydim, butylate, cafenstrole, caloxydim, carbetamide, carfentrazone (-ethyl), chlomethoxyfen, chloramben, chloridazon, chlorimuron (-ethyl), chlomitrofen, chlorsulfuron, chlortoluron, cinidon (-ethyl), cinmethylin, cinosulfuron, clefoxydim, clethodim, clodinafop (-propargyl), clomazone, clomeprop, clopyralid, clopyrasulfuron (-methyl), cloransulam (-methyl), cumyluron, cyanazine, cybutryne, cycloate, cyclosulfamuron, cycloxydim, cyhalofop (-butyl), 2,4-D, 2,4-DB, desmedipham, diallate, dicamba, dichlorprop (-P), diclofop (-methyl), diclosulam, diethatyl (-ethyl), difenzoquat, diflufenican, diflufenzopyr, dimefuron, dimepiperate, dimethachlor, dimethametryn, dimethenamid, dimexyflam, dinitramine, diphenamid, diquat, dithiopyr, diuron, dymron, epropodan, EPTC, esprocarb, ethalfluralin, ethametsulfuron (-methyl), ethofumesate, ethoxyfen, ethoxysulfuron, etobenzanid, fenoxaprop (-P-ethyl), fentrazamide, flamprop (-isopropyl, -isopropyl-L, -methyl), flazasulfuron, florasulam, fluazifop (-P-butyl), fluazolate, flucarbazone (-sodium), flufenacet, flufenpyr, flumetsulam, flumiclorac (-pentyl), flumioxazin, flumipropyn, flumetsulam, fluometuron, fluorochloridone, fluoroglycofen (-ethyl), flupoxam, flupropacil, flurpyrsulfuron (-methyl, -sodium), flurenol (-butyl), fluridone, fluroxypyr (-butoxypropyl, -meptyl), flurprimidol, flurtamone, fluthiacet (-methyl), fluthiamide, fomesafen, foramsulfuron, glufosinate (-ammonium), glyphosate (-isopropylammonium), halosafen, haloxyfop (-ethoxyethyl, -P-methyl), hexazinone, imazamethabenz (-methyl), imazamethapyr, imazamox, imazapic, imazapyr, imazaquin, imazethapyr, imazosulfuron, iodosulfuron (-methyl, -sodium), ioxynil, isopropalin, isoproturon, isouron, isoxaben, isoxachlortole, isoxaflutole, isoxapyrifop, ketospiradox, lactofen, lenacil, linuron, MCPA, mecoprop, mefenacet, mesotrione, metamitron, metazachlor, methabenzthiazuron, metobenzuron, metobromuron, (alpha-) metolachlor, metosulam, metoxuron, metribuzin, metsulfuron (-methyl), molinate, monolinuron, naproanilide, napropamide, neburon, nicosulfuron, norflurazon, orbencarb, oryzalin, oxadiargyl, oxadiazon, oxasulfuron, oxaziclomefone, oxyfluorfen, paraquat, pelargonic acid, pendimethalin, pendralin, penoxysulam, pentoxazone, pethoxamid, phenmedipham, picolinafen, piperophos, pretilachlor, primisulfuron (-methyl), profluazol, profoxydim, prometryn, propachlor, propanil, propaquizafop, propisochlor, propoxycarbazone (-sodium), propyzamide, prosulfocarb, prosulfuron, pyraflufen (-ethyl), pyrazogyl, pyrazolate, pyrazosulfuron (-ethyl), pyrazoxyfen, pyribenzoxim, pyributicarb, pyridate, pyridatol, pyriftalid, pyriminobac (-methyl), pyrithiobac (-sodium), quinchlorac, quinmerac, quinoclamine, quizalofop (-P-ethyl, -P-tefuryl), rimsulfuron, sethoxydim, simazine, simetryn, sulcotrione, sulfentrazone, sulfometuron (-methyl), sulfosate, sulfosulfuron, tebutam, tebuthiuron, tepraloxydim, terbuthylazine, terbutryn, thenylchlor, thiafluamide, thiazopyr, thidiazimin, thifensulfuron (-methyl), thiobencarb, tiocarbazil, tralkoxydim, triallate, triasulfuron, tribenuron (-methyl), triclopyr, tridiphane, trifluralin, trifloxysulfuron, triflusulfuron (-methyl), tritosulfuron.

Furthermore suitable for the mixtures are known safeners, for example AD-67, BAS-145138, benoxacor, cloquintocet (-mexyl), cyometrinil, 2,4-D, DKA-24, dichlormid, dymron, fenclorim, fenchlorazol (-ethyl), flurazole, fluxofenim, furilazole, isoxadifen (-ethyl), MCPA, mecoprop (-P), mefenpyr (-diethyl), MG-191, oxabetrinil, PPG-1292, R-29148.

A mixture with other known active compounds, such as fungicides, insecticides, acaricides, nematicides, bird repellents, plant nutrients and agents which improve soil structure, is also possible.

The active compounds can be used as such, in the form of their formulations or in the use forms prepared therefrom by further dilution, such as ready-to-use solutions, suspensions, emulsions, powders, pastes and granules. They are used in a customary manner, for example by watering, spraying, atomizing or broadcasting.

The active compounds according to the invention can be applied both before and after emergence of the plants. They can also be incorporated into the soil before sowing.

The amount of active compound used can vary within a relatively wide range. It depends essentially on the nature of the desired effect. In general, the amounts used are between 1 g and 10 kg of active compound per hectare of soil surface, preferably between 5 g and 5 kg per ha.

As already mentioned above, it is possible to treat all plants and their parts according to the invention. In a preferred embodiment, wild plant species and cultivars, or those obtained by conventional biological breeding, such as crossing or protoplast fusion, and parts thereof, are treated. In a further preferred embodiment, transgenic plants and cultivars obtained by genetic engineering, if appropriate in combination with conventional methods (Genetically Modified Organisms), and parts thereof are treated. The term “parts” or “parts of plants” or “plant parts” has been explained above.

Particularly preferably, plants of the cultivars which are in each case commercially available or in use are treated according to the invention. Cultivars are to be understood as meaning plants having certain properties (“traits”) and which have been obtained by conventional breeding, by mutagenesis or by recombinant DNA techniques. They can be varieties, bio- or genotypes.

Depending on the plant species or cultivars, their location and growth conditions (soils, climate, vegetation period, diet), the treatment according to the invention may also result in superadditive (“synergistic”) effects. Thus, for example, reduced application rates and/or a widening of the activity spectrum and/or an increase in the activity of the substances and compositions which can be used according to the invention also in combination with other agrochemically active compounds, better plant growth, increased tolerance to high or low temperatures, increased tolerance to drought or to water or soil salt content, increased flowering performance, easier harvesting, accelerated maturation, higher harvest yields, better quality and/or a higher nutritional value of the harvested products, better storage stability and/or processability of the harvested products are possible which exceed the effects which were actually to be expected.

The transgenic plants or cultivars (i.e. those obtained by genetic engineering) which are preferably treated according to the invention include all plants which, in the genetic modification, received genetic material which imparted particularly advantageous useful properties (“traits”) to these plants. Examples of such properties are better plant growth, increased tolerance to high or low temperatures, increased tolerance to drought or to water or soil salt content, increased flowering performance, easier harvesting, accelerated maturation, higher harvest yields, better quality and/or a higher nutritional value of the harvested products, better storage stability and/or processability of the harvested products. Further and particularly emphasized examples of such properties are a better defence of the plants against animal and microbial pests, such as against insects, mites, phytopathogenic fungi, bacteria and/or viruses, and also increased tolerance of the plants to certain herbicidally active compounds. Examples of transgenic plants which may be mentioned are the important crop plants, such as cereals (wheat, rice), maize, soya beans, potatoes, cotton, oilseed rape and also fruit plants (with the fruits apples, pears, citrus fruits and grapes), and particular emphasis is given to maize, soya beans, potatoes, cotton and oilseed rape. Traits that are emphasized are in particular increased defence of the plants against insects by toxins formed in the plants, in particular those formed in the plants by the genetic material from Bacillus thuringiensis (for example by the genes CryIA(a), CryIA(b), CryIA(c), CryIIA, CryIIIA, CryIIIB2, Cry9c Cry2Ab, Cry3Bb and CryIF and also combinations thereof) (hereinbelow referred to as “Bt plants”). Traits that are also particularly emphasized are the increased defence of the plants to fungi, bacteria and viruses by systemic acquired resistance (SAR), systemin, phytoalexins, elicitors and resistance genes and correspondingly expressed proteins and toxins. Traits that are furthermore particularly emphasized are the increased tolerance of the plants to certain herbicidally active compounds, for example imidazolinones, sulphonylureas, glyphosate or phosphinotricin (for example the “PAT” gene). The genes which impart the desired traits in question can also be present in combination with one another in the transgenic plants. Examples of “Bt plants” which may be mentioned are maize varieties, cotton varieties, soya bean varieties and potato varieties which are sold under the trade names YIELD GARD® (for example maize, cotton, soya beans), KnockOut® (for example maize), StarLink® (for example maize), Bollgard® (cotton), Nucotn® (cotton) and NewLeaf® (potato). Examples of herbicide-tolerant plants which may be mentioned are maize varieties, cotton varieties and soya bean varieties which are sold under the trade names Roundup Ready® (tolerance to glyphosate, for example maize, cotton, soya bean), Liberty Link® (tolerance to phosphinotricin, for example oilseed rape), IMI® (tolerance to imidazolinones) and STS® (tolerance to sulphonylurea, for example maize). Herbicide-resistant plants (plants bred in a conventional manner for herbicide tolerance) which may be mentioned include the varieties sold under the name Clearfield® (for example maize). Of course, these statements also apply to cultivars having these genetic traits or genetic traits still to be developed, which plants will be developed and/or marketed in the future.

The plants listed can be treated according to the invention in a particularly advantageous manner with the compounds of the general formula (I) or the active compound mixtures according to the invention where in addition to the effective control of the weeds, the abovementioned synergistic effects with the transgenic plants or plant cultivators occur. The preferred ranges stated above for the active compounds or mixtures also apply to the treatment of these plants. Particular emphasis is given to the treatment of plants with the compounds or mixtures specifically mentioned in the present text.

The preparation and the use of the active compounds according to the invention is illustrated by the examples below.

Preparation Examples:

### EXAMPLE 1

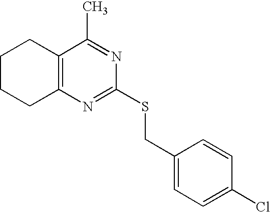 (Process (a))

4.0 g (18.5 mmol) of 4-methyl-5,6,7,8-tetrahydro-2-quinazolinethiol are dissolved in 12 ml of chloroform and, at room temperature (about 20° C.), 1.87 g (18.5 mmol) of triethylamine and 2.98 g (18.5 mmol) of 4-chlorobenzyl chloride and, finally, 3.74 g (27 mmol) of triethylamine are added dropwise successively. The reaction mixture is then stirred at 50° C. for 12 hours and then, at room temperature, washed with water, dried with sodium sulphate and filtered. From the filtrate, volatile components are distilled off under reduced pressure. The residue is digested with petroleum ether, and the resulting crystalline product is isolated by filtration with suction.

This gives 3.3 g (59% of theory) of 2-(4-chlorobenzylthio)-4-methyl-5,6,7,8-tetrahydroquinazoline of melting point 48° C.

### EXAMPLE 2

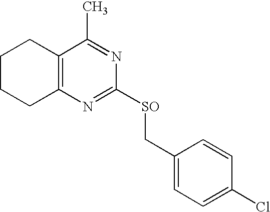 (Process (b))

1.00 g (3.3 mmol) of 2-(4-chlorobenzylthio)-4-methyl-5,6,7,8-tetrahydroquinazoline are dissolved in 20 ml of methylene chloride and, at 0° C., 0.80 g (3.3 mmol) of m-chloroperbenzoic acid is added. The reaction mixture is stirred at 0° C. for 60 minutes and then diluted with 80 ml of methylene chloride, extracted successively with saturated aqueous sodium bicarbonate solution and water and then dried with sodium sulphate. From the filtrate, the solvent is distilled off under reduced pressure, the residue is digested with petroleum ether and the resulting crystalline product is isolated by filtration with suction.

This gives 0.60 g (57% of theory) of 2-(4-chlorobenzylsulphinyl)-4-methyl-5,6,7,8-tetrahydroquinazoline of melting point 127° C.

### EXAMPLE 3

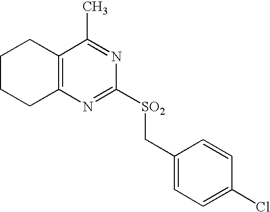 (Process (b))

1.00 g (3.3 mmol) of 2-(4-chlorobenzylthio)-4-methyl-5,6,7,8-tetrahydroquinazoline are dissolved in 30 ml of chloroform and, at room temperature (about 20° C.), a spatula tip (about 0.1 mg) of ammonium molybdate, 0.68 g (13 mmol) of formic acid (85% strength) and 1.24 g (13 mmol) of hydrogen peroxide (35% strength) are added successively. The reaction mixture is then stirred at room temperature for 12 hours and then diluted with 50 ml of methylene chloride, extracted successively with saturated aqueous sodium bicarbonate solution and water and then dried using sodium sulphate. From the filtrate, the solvent is distilled off under reduced pressure, the residue is digested with petroleum ether and the resulting crystalline product is isolated by filtration with suction.

This gives 0.47 g (42% of theory) of 2-(4-chlorobenzylsulphonyl)-4-methyl-5,6,7,8-tetrahydroquinazoline of melting point 151° C.

Analogously to Preparation Examples 1, 2 and 3, and in accordance with the general description of the processes according to the invention, it is also possible to prepare, for example, the compounds of the general formula (I) listed in Table 1 below.

TABLE 1

| | |
|---|---|
| | (I) |
| 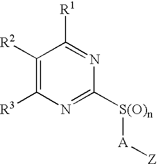 | |

Examples of compounds of the formula (I)

| Ex. No. | n | A | R¹ | R² | R³ | Z | Melting point (° C.) |
|---|---|---|---|---|---|---|---|
| 4 | 2 | CH₂ | CH₃ | R² + R³: (CH₂)₃ | | 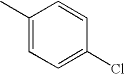 | 179 |
| 5 | 0 | CH₂ | CH₃ | R² + R³: (CH₂)₃ | | 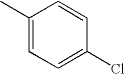 | 35 |
| 6 | 0 | CH₂ | H | C₂H₅ | CH₃ | 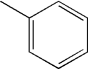 | (amorphous) |
| 7 | 0 | CH₂ | H | C₂H₅ | CH₃ | 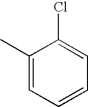 | 47 |
| 8 | 0 | CH₂ | H | C₂H₅ | CH₃ | 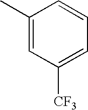 | 32 |
| 9 | 0 | CH₂ | H | C₂H₅ | CH₃ | 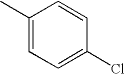 | 47 |
| 10 | 0 | CH₂ | H | C₂H₅ | CH₃ | 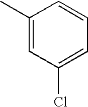 | (amorphous) |
| 11 | 0 | CH₂ | CH₃ | CH₃ | CH₃ | 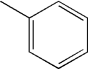 | 103 |
| 12 | 0 | CH₂ | CH₃ | CH₃ | CH₃ | 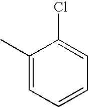 | 66 |
| 13 | 0 | CH₂ | CH₃ | CH₃ | CH₃ | 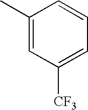 | 65 |
| 14 | 0 | CH₂ | CH₃ | CH₃ | CH₃ | 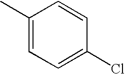 | 66 |
| 15 | 1 | CH₂ | H | C₂H₅ | CH₃ | 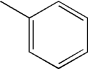 | (amorphous) |
| 16 | 1 | CH₂ | H | C₂H₅ | CH₃ | 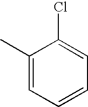 | 45 |
| 17 | 1 | CH₂ | H | C₂H₅ | CH₃ | 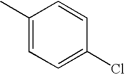 | 90 |
| 18 | 1 | CH₂ | H | C₂H₅ | CH₃ | 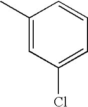 | 59 |
| 19 | 1 | CH₂ | CH₃ | CH₃ | CH₃ | 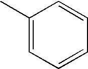 | 99 |
| 20 | 1 | CH₂ | CH₃ | CH₃ | CH₃ | 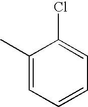 | 113 |
| 21 | 1 | CH₂ | CH₃ | CH₃ | CH₃ | 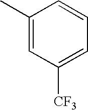 | 43 |
| 22 | 1 | CH₂ | CH₃ | CH₃ | CH₃ | 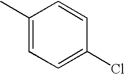 | 164 |
| 23 | 1 | CH₂ | H | C₃H₇-n | H | 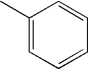 | (amorphous) |
| 24 | 1 | CH₂ | H | C₃H₇-n | H | 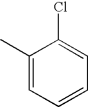 | (amorphous) |
| 25 | 1 | CH₂ | H | C₃H₇-n | H | 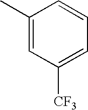 | (amorphous) |
| 26 | 1 | CH₂ | H | C₃H₇-n | H | 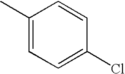 | 54 |
| 27 | 0 | CH₂ | Cl | 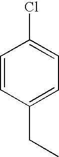 | Cl | 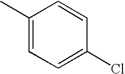 | 153 |
| 28 | 0 | CH₂ | H | C₃H₇-n | H | 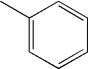 | (amorphous) |
| 29 | 1 | CH₂ | H | C₂H₅ | CH₃ | 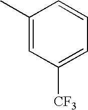 | 47 |
| 30 | 2 | CH₂ | H | C₂H₅ | CH₃ | 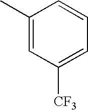 | 76 |
| 31 | 0 | CH₂ | H | C₃H₇-n | H | 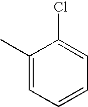 | (amorphous) |
| 32 | 0 | CH₂ | H | C₃H₇-n | H | 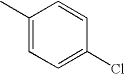 | 65 |
| 33 | 0 | CH₂ | H | C₃H₇-n | H | 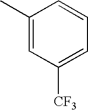 | (amorphous) |
| 34 | 2 | CH₂ | H | C₂H₅ | CH₃ | 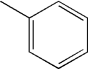 | 90 |
| 35 | 2 | CH₂ | H | C₂H₅ | CH₃ | 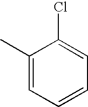 | 103 |
| 36 | 2 | CH₂ | H | C₂H₅ | CH₃ | 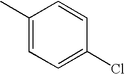 | 75 |
| 37 | 2 | CH₂ | H | C₂H₅ | CH₃ | 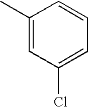 | 108 |
| 38 | 2 | CH₂ | CH₃ | CH₃ | CH₃ | 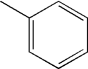 | 158 |
| 39 | 2 | CH₂ | CH₃ | CH₃ | CH₃ | 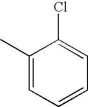 | 146 |
| 40 | 2 | CH₂ | CH₃ | CH₃ | CH₃ | 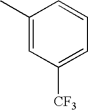 | 108 |
| 41 | 2 | CH₂ | CH₃ | CH₃ | CH₃ | 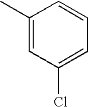 | 118 |
| 42 | 2 | CH₂ | H | C₃H₇-n | H | 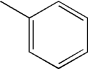 | (amorphous) |
| 43 | 2 | CH₂ | H | C₃H₇-n | H | 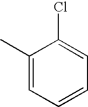 | (amorphous) |
| 44 | 2 | CH₂ | H | C₃H₇-n | H | 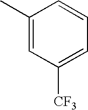 | 96 |
| 45 | 2 | CH₂ | H | C₃H₇-n | H | 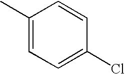 | 81 |
| 46 | 0 | CH₂ | CH₃ | C₂H₅ | CH₃ | 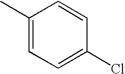 | 60 |
| 47 | 0 | CH₂ | CH₃ | C₃H₇-n | CH₃ | 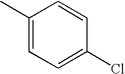 | 30 |
| 48 | 0 | CH₂ | CH₃ | C₄H₉-n | CH₃ | 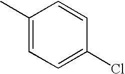 | 31 |
| 49 | 1 | CH₂ | CH₃ | C₃H₇-n | CH₃ | 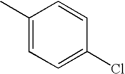 | 143 |
| 50 | 2 | CH₂ | CH₃ | C₃H₇-n | CH₃ | 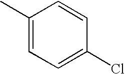 | 92 |
| 51 | 2 | CH₂ | CH₃ | C₄H₉-n | CH₃ | 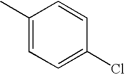 | 71 |
| 52 | 1 | CH₂ | CH₃ | C₂H₅ | CH₃ | 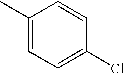 | 109 |
| 53 | 2 | CH₂ | CH₃ | C₂H₅ | CH₃ | 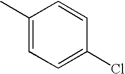 | 115 |
| 54 | 1 | CH₂ | CH₃ | C₄H₉-n | CH₃ | 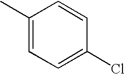 | 76 |
| 55 | 1 | CH₂ | CH₃ | R² + R³: (CH₂)₃ | | 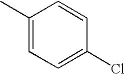 | 170 |
| 56 | 1 | CH₂ | CH₃ | CH₃ | CH₃ | 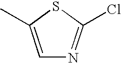 | 98 |
| 57 | 1 | CH₂ | CH₃ | CH₃ | CH₃ | 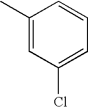 | 87 |
| 58 | 1 | CH₂ | CH₃ | CH₃ | CH₃ | 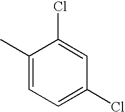 | 180 |
| 59 | 2 | CH₂ | CH₃ | CH₃ | CH₃ | 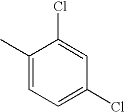 | 146 |
| 60 | 2 | CH₂ | CH₃ | CH₃ | CH₃ | 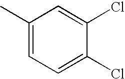 | 162 |
| 61 | 0 | CH(CH₃) | CH₃ | CH₃ | CH₃ | 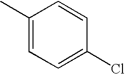 | 123 (racemate) |
| 62 | 0 | CH₂ | CH₃ | CH₃ | CH₃ | 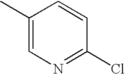 | 80 |
| 63 | 0 | CH₂ | CH₃ | CH₃ | CH₃ | 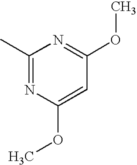 | 148 |
| 64 | 0 | CH₂ | CH₃ | CH₃ | CH₃ | 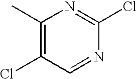 | 110 |
| 65 | 1 | CH(CH₃) | CH₃ | CH₃ | CH₃ | 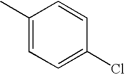 | 123 (racemate) |
| 66 | 2 | CH(CH₃) | CH₃ | CH₃ | CH₃ | 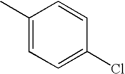 | 123 (racemate) |
| 67 | 1 | CH₂ | CH₃ | CH₃ | CH₃ | 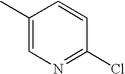 | 153 |
| 68 | 2 | CH₂ | CH₃ | CH₃ | CH₃ | 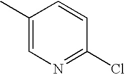 | (amorphous) |
| 69 | 2 | CH₂ | CH₃ | CH₃ | CH₃ | 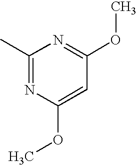 | 113 |
| 70 | 2 | CH₂ | CH₃ | CH₃ | CH₃ | 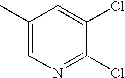 | 150 |
| 71 | 2 | CH₂ | CH₃ | CH₃ | CH₃ | 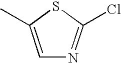 | 107 |
| 72 | 2 | CH₂ | CH₃ | CH₃ | CH₃ | 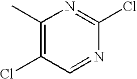 | 103 |
| 73 | 1 | CH₂ | CH₃ | CH₃ | CH₃ | 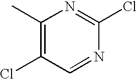 | 103 |
| 74 | 0 | CH₂ | CH₃ | CH₃ | CH₃ | 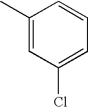 | 62 |
| 75 | 0 | CH₂ | CH₃ | CH₃ | CH₃ | 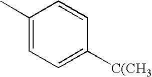 | 65 |
| 76 | 0 | CH₂ | CH₃ | CH₃ | CH₃ | 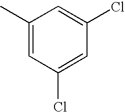 | 112 |
| 77 | 0 | CH₂ | CH₃ | CH₃ | CH₃ | 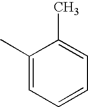 | 126 |
| 78 | 0 | CH₂ | CH₃ | CH₃ | CH₃ | 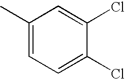 | 91 |
| 79 | 0 | CH₂ | CH₃ | CH₃ | CH₃ | 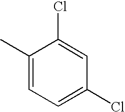 | 60 |
| 80 | 0 | CH₂ | CH₃ | CH₃ | CH₃ | 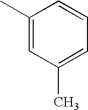 | 35 |
| 81 | 0 | CH₂ | CH₃ | CH₃ | CH₃ | 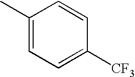 | 65 |
| 82 | 1 | CH₂ | CH₃ | CH₃ | CH₃ | 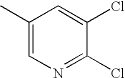 | 102 |
| 83 | 0 | CH₂ | CH₃ | CH₃ | CH₃ | 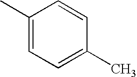 | 80 |
| 84 | 2 | CH₂ | CH₃ | CH₃ | CH₃ | 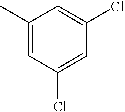 | 148 |
| 85 | 1 | CH₂ | CH₃ | CH₃ | CH₃ | 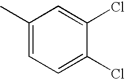 | 134 |
| 86 | 1 | CH₂ | CH₃ | CH₃ | CH₃ | 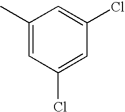 | 130 |
| 87 | 0 | CH₂ | CH₃ | CH₃ | CH₃ | 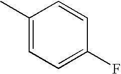 | 78 |
| 88 | 1 | CH₂ | CH₃ | CH₃ | CH₃ | 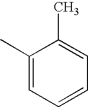 | 147 |
| 89 | 1 | CH₂ | CH₃ | CH₃ | CH₃ | 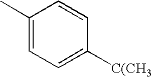 | 149 |
| 90 | 1 | CH₂ | CH₃ | CH₃ | CH₃ | 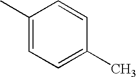 | 101 |
| 91 | 0 | CH₂ | CH₃ | CH₃ | CH₃ | 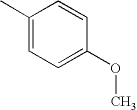 | 52 |
| 92 | 0 | CH₂ | CH₃ | CH₃ | CH₃ | 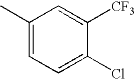 | 98 |
| 93 | 2 | CH₂ | CH₃ | CH₃ | CH₃ | 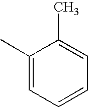 | 134 |
| 94 | 0 | CH₂ | CH₃ | CH₃ | CH₃ | 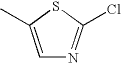 | 77 |
| 95 | 0 | CH₂ | CH₃ | CH₃ | CH₃ | 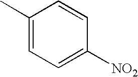 | 111 |
| 96 | 1 | CH₂ | CH₃ | CH₃ | CH₃ | 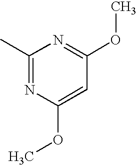 | 126 |
| 97 | 1 | CH₂ | CH₃ | CH₃ | CH₃ | 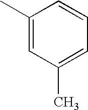 | 166 |
| 98 | 0 | CH₂ | CH₃ | CH₃ | CH₃ | 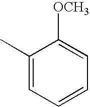 | 83 |
| 99 | 2 | CH₂ | CH₃ | CH₃ | CH₃ | 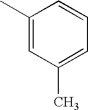 | 95 |
| 100 | 2 | CH₂ | CH₃ | CH₃ | CH₃ | 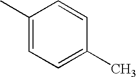 | 90 |
| 101 | 2 | CH₂ | CH₃ | CH₃ | CH₃ | 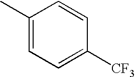 | 108 |
| 102 | 2 | CH₂ | CH₃ | CH₃ | CH₃ | 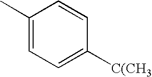 | 190 |
| 103 | 1 | CH₂ | CH₃ | CH₃ | CH₃ | 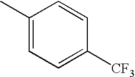 | 190 |
| 104 | 2 | CH₂ | CH₃ | CH₃ | CH₃ | 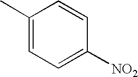 | 203 |
| 105 | 0 | CH₂ | CH₃ | CH₃ | CH₃ | 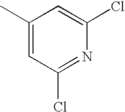 | 118 |
| 106 | 0 | CH₂ | CH₃ | CH₃ | CH₃ | 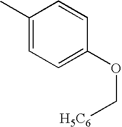 | 48 |
| 107 | 2 | CH₂ | CH₃ | CH₃ | CH₃ | 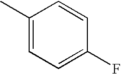 | 128 |
| 108 | 2 | CH₂ | CH₃ | CH₃ | CH₃ | 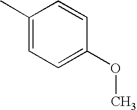 | 116 |
| 109 | 2 | CH₂ | CH₃ | CH₃ | CH₃ | 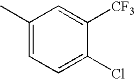 | 183 |
| 110 | 0 | CH₂ | CH₃ | C₂H₅ | CH₃ | 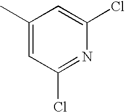 | 94 |
| 111 | 0 | CH₂ | CH₃ | C₃H₇-n | CH₃ | 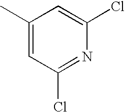 | 80 |
| 112 | 0 | CH₂ | CH₃ | C₄H₉-n | CH₃ | 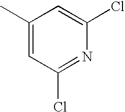 | 80 |
| 113 | 0 | CH₂ | CH₃ | R² + R³: (CH₂)₃ | | 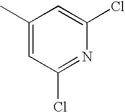 | 105 |
| 114 | 0 | CH₂ | CH₃ | R² + R³: (CH₂)₄ | | 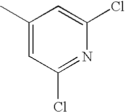 | 64 |
| 115 | 2 | CH₂ | CH₃ | CH₃ | CH₃ | 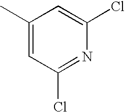 | 153 |
| 116 | 1 | CH₂ | CH₃ | CH₃ | CH₃ | 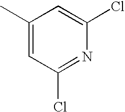 | 154 |
| 117 | 1 | CH₂ | CH₃ | CH₃ | CH₃ | 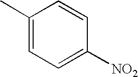 | 181 |
| 118 | 2 | CH₂ | CH₃ | CH₃ | CH₃ | 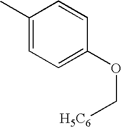 | 134 |
| 119 | 1 | CH₂ | CH₃ | CH₃ | CH₃ | 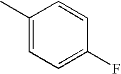 | 117 |
| 120 | 1 | CH₂ | CH₃ | CH₃ | CH₃ | 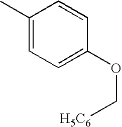 | 104 |
| 121 | 2 | CH₂ | CH₃ | R² + R³: (CH₂)₃ | | 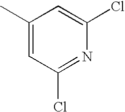 | 166 |
| 122 | 2 | CH₂ | CH₃ | R² + R³: (CH₂)₄ | | 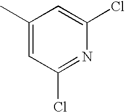 | 148 |
| 123 | 1 | CH₂ | CH₃ | CH₃ | CH₃ | 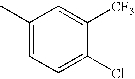 | 171 |
| 124 | 1 | CH₂ | CH₃ | R² + R³: (CH₂)₃ | | 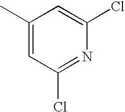 | 166 |
| 125 | 1 | CH₂ | CH₃ | R² + R³: (CH₂)₄ | | 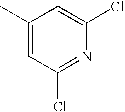 | 143 |
| 126 | 2 | CH₂ | CH₃ | C₃H₇-n | CH₃ | 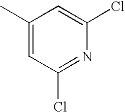 | 117 |
| 127 | 2 | CH₂ | CH₃ | C₄H₉-n | CH₃ | 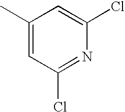 | 84 |
| 128 | 2 | CH₂ | CH₃ | C₂H₅ | CH₃ | 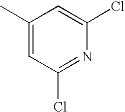 | 105 |
| 129 | 2 | CH₂ | CH₃ | CH₃ | CH₃ | 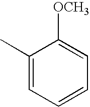 | 103 |
| 130 | 1 | CH₂ | CH₃ | C₂H₅ | CH₃ | 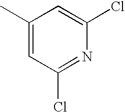 | 107 |
| 131 | 1 | CH₂ | CH₃ | C₄H₉-n | CH₃ | 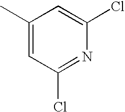 | 89 |
| 132 | 1 | CH₂ | CH₃ | CH₃ | CH₃ | 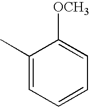 | 154 |
| 133 | 1 | CH₂ | CH₃ | C₃H₇-n | CH₃ | 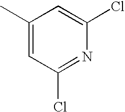 | 91 |
| 134 | 0 | CH₂ | CH₃ | CH₃ | CH₃ | 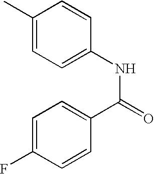 | 209 |
| 136 | 0 | CH₂ | CH₃ | CH₃ | CH₃ | 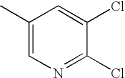 | 105 |
| 137 | 0 | CH₂ | CH₃ | CH₃ | CH₃ | 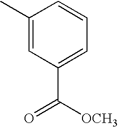 | 64 |
| 138 | 0 | CH₂ | CH₃ | CH₃ | CH₃ | 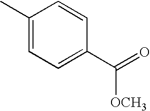 | 67 |
| 139 | 0 | CH₂ | CH₃ | CH₃ | CH₃ | 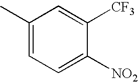 | 75 |
| 140 | 2 | CH₂ | CH₃ | CH₃ | CH₃ | 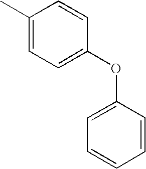 | 98 |
| 141 | 1 | CH₂ | CH₃ | CH₃ | CH₃ | 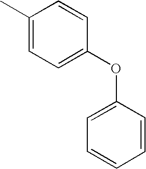 | 110 |
| 142 | 1 | CH₂ | CH₃ | CH₃ | CH₃ | 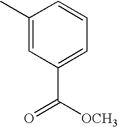 | (oil) |
| 144 | 2 | CH₂ | CH₃ | CH₃ | CH₃ | 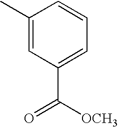 | 76 |
| 145 | 2 | CH₂ | CH₃ | CH₃ | CH₃ | 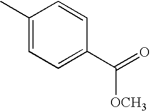 | (oil) |
| 146 | 1 | CH₂ | CH₃ | CH₃ | CH₃ | 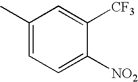 | 161 |
| 147 | 0 | CH₂CH₂ | CH₃ | CH₃ | CH₃ | 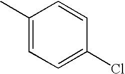 | 224 |
| 148 | 0 | CH₂ | CH₃ | CH₃ | CH₃ | 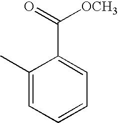 | (oil) |
| 149 | 0 | CH₂ | C₆H₅ | 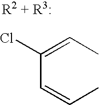 | | 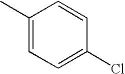 | 93 |
| 150 | 0 | CH₂ | C₆H₅ | 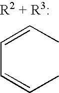 | | 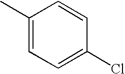 | 92 |
| 151 | 2 | CH₂ | CH₃ | CH₃ | CH₃ | 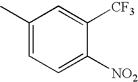 | 176 |
| 152 | 1 | CH₂ | CH₃ | CH₃ | CH₃ | 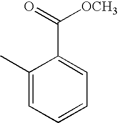 | (oil) |
| 153 | 2 | CH₂ | CH₃ | CH₃ | CH₃ | 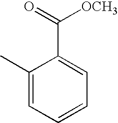 | 88 |
| 154 | 0 | CH₂ | CH₃ | CH₃ | CH₃ | 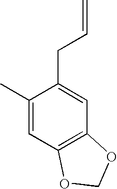 | 72 |
| 155 | 2 | CH₂CH₂ | CH₃ | CH₃ | CH₃ | 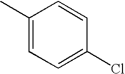 | 118 |
| 156 | 1 | CH₂CH₂ | CH₃ | CH₃ | CH₃ | 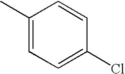 | (oil) |
| 157 | 0 | CH₂ | CH₃ | CH₃ | CH₃ | 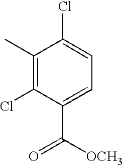 | 59 |
| 158 | 2 | CH₂ | CH₃ | CH₃ | CH₃ | 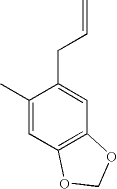 | (oil) |
| 159 | 2 | CH₂ | C₆H₅ | 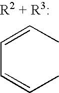 | | 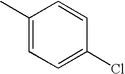 | 105 |
| 160 | 1 | CH₂ | C₆H₅ | 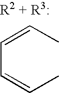 | | 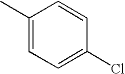 | (oil) |
| 161 | 1 | CH₂ | CH₃ | CH₃ | CH₃ | 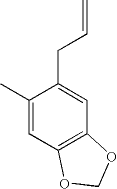 | (oil) |
| 162 | 1 | CH₂ | C₆H₅ | 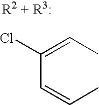 | | 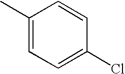 | 129 |
| 163 | 2 | CH₂ | C₆H₅ | 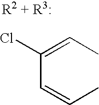 | | 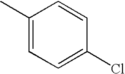 | 197 |
| 164 | 2 | CH₂ | CH₃ | CH₃ | CH₃ | 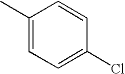 | 143 |
| 165 | 1 | CH₂ | CH₃ | CH₃ | CH₃ | 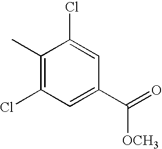 | (oil) |
| 166 | 2 | CH₂ | CH₃ | CH₃ | CH₃ | 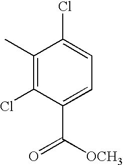 | |
| 167 | 2 | (CH₂)₃ | CH₃ | CH₃ | CH₃ | 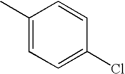 | 81 |
| 168 | 0 | (CH₂)₃ | CH₃ | CH₃ | CH₃ | 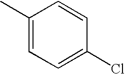 | 65 |
| 169 | 1 | (CH₂)₃ | CH₃ | CH₃ | CH₃ | 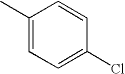 | 72 |
| 170 | 1 | CH₂ | H | CH₃ | CH₃ | 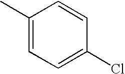 | 104 |
| 171 | 2 | CH₂ | H | CH₃ | CH₃ | 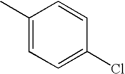 | 78 |

Use Examples:

### EXAMPLE A

Post-Emergence Test

| | |
|---|---|
| Solvent: | 5 parts by weight of acetone |
| Emulsifier: | 1 part by weight of alkylaryl polyglycol ether |

To produce a suitable preparation of active compound, 1 part by weight of active compound is mixed with the stated amount of solvent, the stated amount of emulsifier is added and the concentrate is diluted with water to the desired concentration.

Test plants of a height of 5-15 cm are sprayed with the preparation of active compound such that the particular amounts of active compound desired are applied per unit area. The concentration of the spray liquor is chosen such that the particular amounts of active compound desired are applied in 1000 l of water/ha.

After three weeks, the degree of damage to the plants is rated in % damage in comparison to the development of the untreated control.

The figures denote:
0%=no effect (like untreated control)
100%=total destruction

In this test, for example, the compounds of Preparation Examples 4, 22, 55, 85 and 88 exhibit strong activity against weeds, and some of them are tolerated well by crop plants such as, for example, maize.

### EXAMPLE B

Pre-Emergence Test

| | |
|---|---|
| Solvent: | 5 parts by weight of acetone |
| Emulsifier: | 1 part by weight of alkylaryl polyglycol ether |

To produce a suitable preparation of active compound, 1 part by weight of active compound is mixed with the stated amount of solvent, the stated amount of emulsifier is added and the concentrate is diluted with water to the desired concentration.

Seeds of the test plants are sown in normal soil. After 24 hours, the soil is sprayed with the preparation of active compounds such that the particular amount of active compound desired is applied per unit area. The concentration of active compound in the spray liquor is chosen such that the particular amount of active compound desired is applied in 1000 litres of water per hectare.

After three weeks, the degree of damage to the plants is rated in % damage in comparison to the development of the untreated control. The figures denote:
0%=no effect (like untreated control)
100%=total destruction

In this test, for example, the compounds of the Preparation Examples 4, 20, 22, 41, 55, 58, 60, 85, 88, 93, 97 and 166 exhibit strong activity against weeds, and some of them are tolerated well by crop plants, such as, for example, maize.

TABLE A1
Post-emergence-Test/greenhouse

| Compound of Preparation | application | | | | | |
|---|---|---|---|---|---|---|
| Example No. | rate (g a.i./ha) | maize | Sorghum | Amaranthus | Polygonum | Veronica |
| (4) | 2000 | 0 | 80 | 100 | 80 | 80 |

TABLE A2
Post-emergence-Test/greenhouse

| Compound of Preparation | application | | | | | | |
|---|---|---|---|---|---|---|---|
| Example No. | rate (g a.i./ha) | Setaria | Abutilon | Amaranthus | Galium | Ipomoea | Sinapis |
| (22) | 2000 | 95 | 100 | 100 | 95 | 90 | — |
| (55) | 2000 | 80 | 95 | 100 | 80 | — | 80 |
| (85) | 2000 | 90 | — | 90 | 95 | — | 70 |

TABLE A3
Post-emergence-Test/greenhouse

| Compound of Preparation | application | | | | | | |
|---|---|---|---|---|---|---|---|
| Example No. | rate (g a.i./ha) | maize | Setaria | Abutilon | Amaranthus | Galium | Sinapis |
| (88) | 1000 | 10 | 90 | 80 | 100 | 90 | 80 |

TABLE B1
Pre-emergence-Test/greenhouse

| Compound of Preparation | application | | | | | |
|---|---|---|---|---|---|---|
| Example No. | rate (g a.i./ha) | Setaria | Amaranthus | Polygonum | Veronica | Viola |
| (4) | 4000 | 70 | 100 | 95 | 90 | 80 |

TABLE B2
Pre-emergence-Test/greenhouse

| Compound of Preparation | application | | | | | | |
|---|---|---|---|---|---|---|---|
| Example No. | rate (g a.i./ha) | maize | Setaria | Amaranthus | Galium | Ipomoea | Sinapis |
| (20) | 2000 | 0 | 95 | 90 | 90 | 80 | — |
| (22) | 2000 | 0 | 100 | 100 | 90 | 80 | 80 |

TABLE B3
Pre-emergence-Test/greenhouse

| Compound of Preparation | application | | | | | |
|---|---|---|---|---|---|---|
| Example No. | rate (g a.i./ha) | maize | Setaria | Abutilon | Amaranthus | Galium |
| (55) | 1000 | 10 | 90 | 70 | 100 | 80 |

TABLE B4
Pre-emergence-Test/greenhouse

| Compound of Preparation | application | | Avena | | | |
|---|---|---|---|---|---|---|
| Example No. | rate (g a.i./ha) | Alopecurus | fatua | Setaria | Amaranthus | Galium |
| (41) | 2000 | 90 | 80 | 100 | 100 | 80 |

TABLE B5
Pre-emergence-Test/greenhouse

| Compound of Preparation | application | | | | | |
|---|---|---|---|---|---|---|
| Example No. | rate (g a.i./ha) | Alopecurus | Setaria | Amaranthus | Galium | Ipomoea |
| (166) | 2000 | 70 | 95 | 100 | 95 | — |
| (60) | 2000 | 80 | 100 | 100 | 90 | 70 |

TABLE B6
Pre-emergence-Test/greenhouse

| Compound of Preparation | application | | | | | |
|---|---|---|---|---|---|---|
| Example No. | rate (g a.i./ha) | Setaria | Abutilon | Amaranthus | Galium | Ipomoea |
| (58) | 2000 | 90 | 100 | 100 | 90 | 80 |

TABLE B7
Pre-emergence-Test/greenhouse

| Compound of Preparation | application | | | | | |
|---|---|---|---|---|---|---|
| Example No. | rate (g a.i./ha) | Alopecurus | Setaria | Abutilon | Amaranthus | Galium |
| (85) | 2000 | 80 | 100 | 100 | 100 | 100 |

TABLE B8
Pre-emergence-Test/greenhouse

| Compound of Preparation | application | | | | | | |
|---|---|---|---|---|---|---|---|
| Example No. | rate (g a.i./ha) | Alopecurus | Setaria | Abutilon | Amaranthus | Galium | Sinapis |
| (88) | 2000 | 80 | 100 | 80 | 100 | 80 | 90 |

TABLE B9
Pre-emergence-Test/greenhouse

| Compound of Preparation | application | | | | | |
|---|---|---|---|---|---|---|
| Example No. | rate (g a.i./ha) | Avena fatua | Setaria | Amaranthus | Galium | Ipomoea |
| (97) | 2000 | 80 | 95 | 90 | 80 | — |
| (93) | 2000 | 80 | 80 | 100 | 90 | 95 |

1-9. (Canceled).

10. A compound of formula (I) 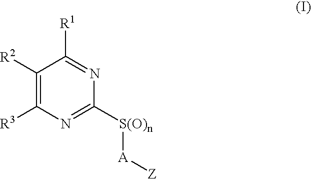
in which
n represents the number 0, 1, or 2,
A represents straight-chain or branched alkanediyl having 1 to 6 carbon atoms,
R¹ represents hydrogen, halogen, optionally halogen- or C₁-C₄-alkoxy-substituted alkyl having 1 to 6 carbon atoms, or optionally halogen- or C₁-C₄-alkyl-substituted phenyl,
R² represents optionally halogen- or C₁-C₄-alkoxy-substituted alkyl having 1 to 6 carbon atoms; represents optionally halogen- or C₁-C₄-alkyl-substituted cycloalkyl having 3 to 8 carbon atoms; or represents optionally halogen- or C₁-C₄-alkyl-substituted phenyl-C₁-C₄-alkyl, or together with R¹ or together with R³ represents optionally C₁-C₄-alkyl-substituted alkanediyl having 3 to 5 carbon atoms or an optionally halogen- or C₁-C₄-alkyl-substituted benzo group,
R³ represents hydrogen, halogen, optionally halogen- or C₁-C₄-alkoxy-substituted alkyl having 1 to 6 carbon atoms, or optionally halogen- or C₁-C₄-alkyl-substituted phenyl, and
Z represents optionally substituted phenyl, naphthyl, pyridinyl, pyrimidinyl, furyl, thienyl, oxazolyl, or thiazolyl, wherein the substituents are nitro, cyano, carboxyl, carbamoyl, thiocarbamoyl, or halogen; optionally cyano-, carboxyl-, carbamoyl-, thiocarbamoyl-, halogen-, C₁-C₄-alkoxy-, C₁-C₄-alkylthio-, C₁-C₄-alkylsulphinyl-, or C₁-C₄-alkylsulphonyl-substituted alkyl, alkoxy, alkoxycarbonyl, alkylthio, alkylsulphinyl, alkylsulphonyl, alkylamino, alkylaminocarbonyl, or alkylcarbonylamino having in each case 1 to 6 carbon atoms in the alkyl groups; optionally halogen-substituted alkylenedioxy having 1 or 2 carbon atoms; dialkylamino, dialkylaminocarbonyl, or dialkylaminosulphonyl having in each case 1 to 4 carbon atoms in the alkyl groups; or optionally nitro-, cyano-, carboxyl-, carbamoyl-, thiocarbamoyl-, halogen-, C₁-C₄-alkyl-, C₁-C₄-haloalkyl-, C₁-C₄-alkoxy-, C₁-C₄-haloalkoxy-, C₁-C₄-alkylthio-, C₁-C₄-haloalkylthio-, C₁-C₄-alkylsulphinyl-, C₁-C₄-haloalkylsulphinyl-, C₁-C₄-alkylsulphonyl-, or C₁-C₄-haloalkylsulphonyl-substituted phenyl, phenoxy, phenylthio, phenylsulphinyl, phenylsulphonyl, phenylamino, phenylcarbonylamino, phenyl-C₁-C₄-alkyl, phenyl-C₁-C₄-alkoxy, phenoxy-C₁-C₄-alkyl, phenyl-C₁-C₄-alkylthio, phenyl-C₁-C₄-alkylsulphinyl, phenyl-C₁-C₄-alkylsulphonyl, or phenyl-C₁-C₄-alkylamino,
except for the compounds 2-benzylthio-4-chloro-5-methylpyrimidine, 4-chloro-2-(2,4-dichlorobenzylthio)-5-methylpyrimidine, 4-chloro-2-(chlorobenzylthio)-5-methylpyrimidine and 4-chloro-2-(4-chlorobenzylthio)-5-methylpyrimidine, 4,6-dichloro-5-methyl-2-(naphthalen-2-ylmethylthio)pyrimidine, 5-methyl-2-[(1-methyl-5-nitro-1H-imidazol-2-yl)methylthio]pyrimidine, 2-(thien-2-ylmethylsulphonyl)-5-trifluoromethylpyrimidine, 2-phenylmethylthio-5-trifluoromethylpyrimidine, 2-(thien-2-ylmethylthio)-5-trifluoromethylpyrimidine, 2-phenylmethylsulphonyl-5-trifluoromethylpyrimidine, and 2-(thien-2-ylmethylsulphinyl)-5-trifluoromethylpyrimidine.

11. A compound according to claim 10 in which
n represents the number 0, 1, or 2,
A represents straight-chain or branched alkanediyl having 1 to 4 carbon atoms,
R¹ represents hydrogen, halogen, optionally halogen- or C₁-C₄-alkoxy-substituted alkyl having 1 to 4 carbon atoms, or optionally halogen- or C₁-C₄-alkyl-substituted phenyl,
R² represents optionally halogen- or C₁-C₄-alkoxy-substituted alkyl having 1 to 5 carbon atoms; represents optionally halogen- or C₁-C₃-alkyl-substituted cycloalkyl having 3 to 7 carbon atoms; or represents optionally halogen- or C₁-C₄-alkyl-substituted phenyl-C₁-C₄-alkyl; or R² together with R¹ or R² together with R³ represents optionally C₁-C₃-alkyl-substituted alkanediyl having 3 to 5 carbon atoms or an optionally halogen- or C₁-C₃-alkyl-substituted benzo group,
R³ represents hydrogen, halogen, optionally halogen- or C₁-C₄-alkoxy-substituted alkyl having 1 to 4 carbon atoms, or optionally halogen- or C₁-C₄-alkyl-substituted phenyl, and
Z represents optionally substituted phenyl, naphthyl, pyridinyl, pyrimidinyl, furyl, thienyl, oxazolyl, or thiazolyl, wherein the substituents are nitro, cyano, carboxyl, carbamoyl, thiocarbamoyl, or halogen; optionally cyano-, carboxyl-, carbamoyl-, thiocarbamoyl-, halogen-, C₁-C₄-alkoxy-, C₁-C₄-alkylthio-, C₁-C₄-alkylsulphinyl-, or C₁-C₄-alkylsulphonyl-substituted alkyl, alkoxy, alkoxycarbonyl, alkylthio, alkylsulphinyl, alkylsulphonyl, alkylamino, alkylaminocarbonyl, or alkylcarbonylamino having in each case 1 to 5 carbon atoms in the alkyl groups; optionally halogen-substituted alkylenedioxy having 1 or 2 carbon atoms; dialkylamino, dialkylaminocarbonyl, or dialkylaminosulphonyl having in each case 1 to 3 carbon atoms in the alkyl groups; or optionally nitro-, cyano-, carboxyl-, carbamoyl-, thiocarbamoyl-, halogen-, C₁-C₄-alkyl-, C₁-C₄-haloalkyl-, C₁-C₄-alkoxy-, C₁-C₄-haloalkoxy-, C₁-C₄-alkylthio-, C₁-C₄-haloalkylthio-, C₁-C₄-alkylsulphinyl-, C₁-C₄-haloalkylsulphinyl-, C₁-C₄-alkylsulphonyl-, or C₁-C₄-haloalkylsulphonyl-substituted phenyl, phenoxy, phenylthio, phenylsulphinyl, phenylsulphonyl, phenylamino, phenylcarbonylamino, phenyl-C₁-C₄-alkyl, phenyl-C₁-C₄-alkoxy, phenoxy-C₁-C₄-alkyl, phenyl-C₁-C₄-alkylthio, phenyl-C₁-C₄-alkylsulphinyl, phenyl-C₁-C₄-alkylsulphonyl, or phenyl-C₁-C₄-alkylamino.

12. A compound according to claim 10 in which
A represents methylene, ethane-1,1-diyl (ethylidene), ethane-1,2-diyl (dimethylene), propane-1,1-diyl (propylidene), propane-1,2-diyl, or propane-1,3-diyl (trimethylene),
R¹ represents hydrogen, fluorine, chlorine, bromine, or iodine; represents optionally fluorine-, chlorine-, bromine-, methoxy-, ethoxy-, or n- or isopropoxy-substituted methyl, ethyl, n- or isopropyl, n-, iso-, s-, or t-butyl; or
represents optionally fluorine-, chlorine-, bromine-, methyl-, ethyl-, n- or isopropyl-, or n-, iso-, s-, or t-butyl-substituted phenyl,
R² represents optionally fluorine-, chlorine-, bromine-, methoxy-, ethoxy-, or n- or isopropoxy-substituted methyl, ethyl, n- or isopropyl, n-, iso-, s-, or t-butyl;
represents optionally fluorine-, chlorine-, methyl-, or ethyl-substituted cyclopropyl, cyclobutyl, cyclopentyl, or cyclohexyl; or represents optionally fluorine-, chlorine-, bromine-, methyl-, ethyl-, n- or isopropyl, or n-, iso-, s-, or t-butyl-substituted benzyl or phenylethyl; or R² together with R¹ or R² together with R³ represents optionally methyl- and/or ethyl-substituted propane-1,3-diyl (trimethylene) or butane-1,4-diyl (tetramethylene) or an optionally fluorine-, chlorine-, or methyl-substituted benzo group,
R³ represents hydrogen, fluorine, chlorine, bromine, or iodine; represents optionally fluorine-, chlorine-, bromine-, methoxy-, ethoxy-, or n- or isopropoxy-substituted methyl, ethyl, n- or isopropyl, or n-, iso-, s-, or t-butyl; or represents optionally fluorine-, chlorine-, bromine-, methyl-, ethyl-, n- or isopropyl-, or n-, iso-, s-, or t-butyl-substituted phenyl, and
Z represents optionally substituted phenyl, naphthyl, pyridinyl, pyrimidinyl, furyl, thienyl, oxazolyl, or thiazolyl, wherein the substituents are nitro, cyano, carboxyl, carbamoyl, thiocarbamoyl, fluorine, chlorine, bromine, or iodine;
optionally cyano-, carboxyl-, carbamoyl-, thiocarbamoyl-, fluorine-, chlorine-, methoxy-, ethoxy-, n- or isopropoxy-, methylthio-, ethylthio-, n- or isopropylthio-, methylsulphinyl-, ethylsulphinyl-, methylsulphonyl-, or ethylsulphonyl-substituted methyl, ethyl, n- or isopropyl, n-, iso-, s-, or t-butyl, methoxy, ethoxy, n- or isopropoxy, n-, iso-, s-, or t-butoxy, methoxycarbonyl, ethoxycarbonyl, n- or isopropoxycarbonyl, methylthio, ethylthio, n- or isopropylthio, n-, iso-, s-, or t-butylthio, methylsulphinyl, ethylsulphinyl, n- or isopropyl-sulphinyl, methylsulphonyl, ethylsulphonyl, methylamino, ethylamino, n- or isopropylamino, n-, iso-, s-, or t-butylamino, methylaminocarbonyl, ethylaminocarbonyl, n- or isopropylaminocarbonyl, acetylamino, propionylamino, or n- or isobutyroylamino; optionally fluorine- and/or chlorine-substituted methylenedioxy or ethylenedioxy; dimethylamino, diethylamino, dimethylaminocarbonyl, or dimethylaminosulphonyl; or optionally nitro-, cyano-, carboxyl-, carbamoyl-, thiocarbamoyl-, fluorine-, chlorine-, bromine-, methyl-, ethyl-, n- or isopropyl-, n-, iso-, s-, or t-butyl-, trifluoromethyl-, methoxy-, ethoxy-, n- or isopropoxy-, difluoromethoxy-, trifluoromethoxy-, methylthio-, ethylthio-, n- or isopropylthio-, difluoromethylthio-, trifluoropmethylthio-, methylsulphinyl-, ethylsulphinyl-, trifluoromethylsulphinyl-, methylsulphonyl-, ethylsulphonyl-, or trifluoromethylsulphonyl-substituted phenyl, phenoxy, phenylthio, phenylsulphinyl, phenylsulphonyl, phenylamino, phenylcarbonylamino, benzyl, phenylethyl, phenylmethoxy, phenylethoxy, phenoxymethyl, phenoxyethyl, phenylmethylthio, phenylethylthio, phenylmethylsulphinyl, phenylethylsulphinyl, phenylmethylsulphonyl, phenylethylsulphonyl, phenylmethylamino, or phenylethylamino.

13. A compound according to claim 10 in which
A represents methylene, ethane-1,1-diyl (ethylidene), or ethane-1,2-diyl (dimethylene),
R¹ represents hydrogen, fluorine, chlorine, or bromine; represents optionally fluorine-, chlorine-, bromine-, methoxy-, or ethoxy-substituted methyl, ethyl, n- or isopropyl; or represents optionally fluorine-, chlorine-, bromine-, or methyl-substituted phenyl,
R² represents optionally fluorine-, chlorine-, bromine-, methoxy-, or ethoxy-substituted methyl, ethyl, n- or isopropyl, or n-, iso-, or s-butyl; or represents optionally fluorine-, chlorine-, bromine-, or methyl-substituted benzyl; or R² together with R¹ or R² together with R³ represents optionally methyl- and/or ethyl-substituted propane-1,3-diyl (trimethylene) or butane-1,4-diyl (tetramethylene) or an optionally fluorine-, chlorine-, or methyl-substituted benzo group,
R³ represents hydrogen, fluorine, chlorine, or bromine; represents optionally -fluorine-, chlorine-, bromine-, methoxy-, or ethoxy-substituted methyl, ethyl, or n- or isopropyl; or represents optionally fluorine-, chlorine-, bromine-, or methyl-substituted phenyl, and
Z represents optionally substituted phenyl, pyridinyl, pyrimidinyl, furyl, thienyl, oxazolyl, or thiazolyl, wherein the substituents are nitro, cyano, carboxyl, carbamoyl, thiocarbamoyl, fluorine, chlorine, or bromine; optionally cyano-, carboxyl-, carbamoyl-, thiocarbamoyl-, fluorine-, chlorine-, methoxy-, ethoxy-, n- or isopropoxy-, methylthio-, ethylthio-, n- or isopropylthio-, methylsulphinyl-, ethylsulphonyl-, methylsulphonyl-, or ethylsulphonyl-substituted methyl, ethyl, n- or isopropyl, n-, iso-, s-, or t-butyl, methoxy, ethoxy, n- or isopropoxy, methoxycarbonyl, ethoxycarbonyl, n- or isopropoxycarbonyl, methylthio, ethylthio, n- or isopropylthio, methylsulphinyl, ethylsulphinyl, methylsulphonyl, ethylsulphonyl, methylamino, ethylamino, n- or isopropylamino, methylaminocarbonyl, ethylaminocarbonyl, n- or isopropylaminocarbonyl, acetylamino, or propionylamino; optionally fluorine- and/or chlorine-substituted methylenedioxy or ethylenedioxy; dimethylamino, dimethylaminocarbonyl, or dimethylaminosulphonyl; or optionally nitro-, cyano-, carboxyl-, carbamoyl-, thiocarbamoyl-, fluorine-, chlorine-, bromine-, methyl-, ethyl-, n- or isopropyl-, n-, iso-, s-, or t-butyl-, trifluoromethyl-, methoxy-, ethoxy-, n- or isopropoxy-, difluoromethoxy-, trifluoromethoxy-, methylthio-, ethylthio-, n- or isopropylthio-, difluoromethylthio-, trifluoromethylthio-, methylsulphinyl-, ethylsulphinyl-, trifluoromethylsulphinyl-, methylsulphonyl-, ethylsulphonyl-, or trifluoromethylsulphonyl-substituted phenyl, phenoxy, phenylthio, phenylsulphinyl, phenylsulphonyl, phenylamino, phenylcarbonylamino, benzyl, phenylethyl, phenylmethoxy, phenylethoxy, phenoxymethyl, phenoxyethyl, phenylmethylthio, phenylethylthio, phenylmethylsulphinyl, phenylethylsulphinyl, phenylmethylsulphonyl, phenylethylsulphonyl, phenylmethylamino, or phenylethylamino.

14. A compound according to claim 10 in which Z represents optionally substituted phenyl, pyridinyl, pyrimidinyl, or thiazolyl, where the substituents of Z are as defined in claim 10.

15. A process for preparing a compound according to claim 10 comprising
(a) for compounds of formula (I) in which n represents the number 0, reacting a mercaptopyrimidine of formula (II) 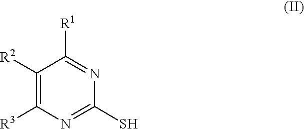
in which R¹, R², and R³ are as defined for formula (I) in claim 10, with a haloalkyl compound of formula (III) 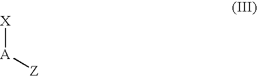
in which
A and Z are as defined for formula (I) in claim 10, and
X represents halogen,
optionally in the presence of a reaction auxiliary and optionally in the presence of a diluent,
or
(b) for compounds of formula (I) in which n represents the number 1 or 2, reacting a substituted pyrimidine of formula (Ia) 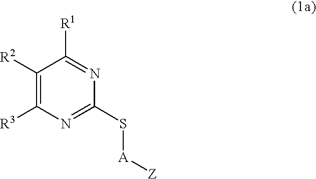
in which A, R¹, R², R³ and Z are as defined for formula (I) in claim 10, with an oxidizing agent,
optionally in the presence of a catalyst and optionally in the presence of a diluent.

16. A herbicidal composition comprising one or more compounds according to claim 10 or a compound excluded in claim 10 and one or more extenders and/or surfactants.

17. A method for controlling unwanted vegetation comprising allowing one or more compounds according to claim 10 or a compound excluded in claim 10 to act on unwanted plants and/or their habitat.
